(19) United States
(12) Patent Application Publication
(10) Pub. No.: US 2020/0068817 A1
(43) Pub. Date: Mar. 5, 2020

(54) METHODS FOR CULTIVATING VANILLA POMPONA AND METHODS OF USE THEREOF

(71) Applicant: Pompona Project LLC, Florissant, MO (US)

(72) Inventors: Annette Renee Crymes, Florissant, MO (US); Isabel Vegas Salazer, Florissant, MO (US); Julian Vasquez Vasquez, Florissant, MO (US); Jose Walter Requejo Cubas, Florissant, MO (US); Henyel Vasquez Labajos, Florissant, MO (US); Kevin Alfred Berdak, Florissant, MO (US)

(21) Appl. No.: 16/485,109

(22) PCT Filed: Feb. 9, 2018
(86) PCT No.: PCT/US2018/017690

§ 371 Date: Aug. 9, 2019

Related U.S. Application Data
(60) Provisional application No. 62/457,616, filed on Feb. 10, 2017.

(51) Int. Cl. A01G 22/63; A23L 27/10; A61K 8/9794; A61K 36/898; A01G 2/10

(52) U.S. Cl. CPC A01G 22/63; A23L 27/10; A61K 8/9794; A01K 47/00; A01G 2/10; A23V 2002/00; A61K 2800/10; A61K 36/898

(57) ABSTRACT

The disclosure provides methods of cultivating Vanilla pompona as well as the curing and use of the cultivated and cured Vanilla pompona seed pods for the production of vanilla products.

### CROSS REFERENCE TO RELATED APPLICATIONS

This application claims the benefit of U.S. provisional application No. 62/457,616, filed on Feb. 10, 2017, the disclosure of which is hereby incorporated by reference in its entirety.

### FIELD OF THE INVENTION

The disclosure provides methods of cultivating Vanilla pompona, curing Vanilla pompona as well as the use of the cultivated Vanilla pompona seed pods for the production of vanilla products.

### BACKGROUND OF THE INVENTION

Natural vanilla is a flavoring derived from orchids of the genus Vanilla (Orchidaceae). Vanilla is a unique spice of great commercial value on the international market. It is one of the most expensive spices in the world, second only to saffron. Despite the expense, vanilla is highly valued for its flavor. Vanilla is widely used in both commercial and domestic foods, perfume manufacture and aromatherapy.

Vanilla planifolia is currently the most popular and widely consumed vanilla on the international market. Vanilla tahitensis is the second most popular. Vanilla pompona (wild Peruvian) is mostly unknown in the flavor and fragrance industry. There is a need in the art for a sustainable cultivation method for Vanilla pompona.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts a diagram of the layout of the cultivation of Vanilla pompona using an Inga tree and further comprising bee hives.

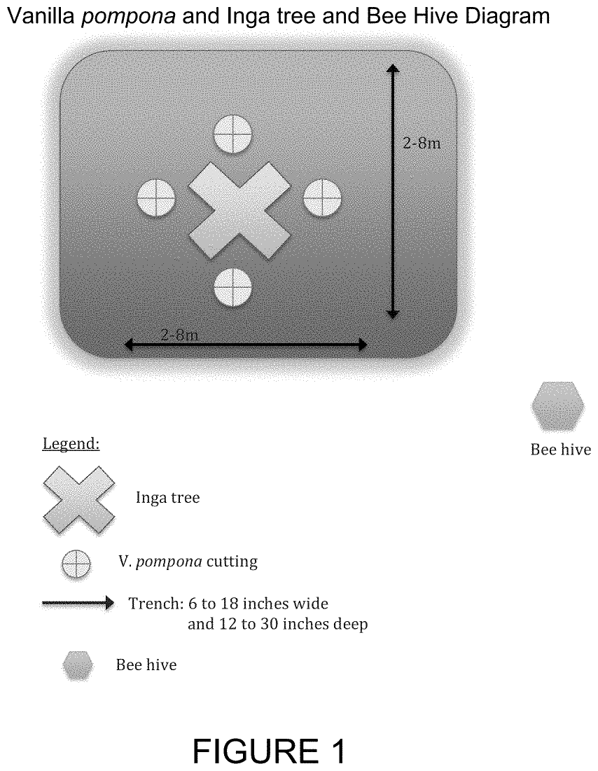

### DETAILED DESCRIPTION OF THE INVENTION

The inventors have a discovered a method of cultivating Vanilla pompona such that the V. pompona plant produces pods within the first year of growth. The ability to efficiently cultivate V. pompona facilitates a new source of vanilla essence/extract. The methods disclosed herein provide a sustainable method of cultivating V. pompona. Various aspects of the methods are described in detail below.

### I. Cultivation

In an aspect, the disclosure provides a method of transplanting Vanilla pompona to produce pods within the first year of growth. The method comprises collecting a cutting between about 150 cm and about 250 cm long, and planting said cutting under conditions suitable to grow V. pompona. The length of the cutting is critical for the production of pods within the first year of growth. The length of the cutting can be about 150 cm to about 250 cm, about 150 cm to about 225 cm, about 150 cm to about 200 cm, about 150 cm to about 175 cm, about 175 cm to about 250 cm, about 175 cm to about 225 cm, about 175 cm to about 200 cm, about 200 cm to about 250 cm, about 200 cm to about 225 cm, or about 225 cm to about 250 cm. Additionally, the length of the cutting can be about 150 cm, about 175 cm, about 200 cm, about 225 cm, or about 250 cm. Further, the length of the cutting can be 150 cm to 250 cm, 150 cm to 225 cm, 150 cm to 200 cm, 150 cm to 175 cm, 175 cm to 250 cm, 175 cm to 225 cm, 175 cm to 200 cm, 200 cm to 250 cm, 200 cm to 225 cm, or 225 cm to 250 cm. Still further, the length of the cutting can be 150 cm, 175 cm, 200 cm, 225 cm, or 250 cm.

In another aspect, the disclosure provides a method of transplanting Vanilla pompona to produce pods within the first year of growth. The method comprises collecting a cutting with a minimum of 12 nodes, and planting said cutting under conditions suitable to grow V. pompona. The number of nodes on the cutting is critical for the production of pods within the first year of growth. The number of nodes on the cutting can be about 12 or more, about 13 or more, about 14 or more, about 15 or more, about 16 or more, about 17 or more, about 18 or more, about 19 or more, about 20 or more, about 21 or more, about 22 or more, about 23 or more, or about 24 or more. Additionally, the number of nodes on the cutting can be about 12, about 13, about 14, about 15, about 16, about 17, about 18, about 19, about 20, about 21, about 22, about 23, or about 24. Further, the number of nodes on the cutting can be 12 or more, 13 or more, 14 or more, 15 or more, 16 or more, 17 or more, 18 or more, 19 or more, 20 or more, 21 or more, 22 or more, 23 or more, or 24 or more. Still further, the number of nodes on the cutting can be 12, 13, 14, 15, 16, 17, 18, 19, 20, 21, 22, 23, or 24.

The cutting can be from a wild V. pompona plant or from a previously cultivated V. pompona plant. A wild V. pompona plant is a plant that grows in nature without the assistance or intervention of humans. A cultivated V. pompona plant is a plant that has been planted, tended to, trained, improved and/or harvested by humans.

Once the cutting has been collected, it is planted under conditions suitable to grow V. pompona. In preferred embodiments, the V. pompona cutting is planted within 7 days following cutting. While the V. pompona cutting may be planted after 7 days following cutting, the viability of the cutting will decrease from thereon. For example, the V. pompona cutting is planted within about 7 days, within about 6 days, within about 5 days, within about 4 days, within about 3 days, within about 2 days, within about 1 day, or the same day following cutting. Additionally, the V. pompona cutting is planted about 7 days, about 6 days, about 5 days, about 4 days, about 3 days, about 2 days, about 1 day, or the same day following cutting. Further, the V. pompona cutting is planted within 7 days, within 6 days, within 5 days, within 4 days, within 3 days, within 2 days, within 1 day, or the same day following cutting. Still further, the V. pompona cutting is planted 7 days, 6 days, 5 days, 4 days, 3 days, 2 days, 1 day, or the same day following cutting. If the cutting is not planted immediately, it should not be allowed to dry out until planted. Prior to planting, leaves from the bottom nodes may be removed. For example, leaves from the bottom 1, 2, 3, or 4 nodes may be removed prior to planting. The base of the cutting may then be inserted into loose soil. The soil may cover the bottom 1, 2, 3, or 4 nodes from which the leaves were removed. The soil may then be packed tightly around the base of the cutting. The cutting may be tied to a support for growth. The soil may be mulched around the planted cutting.

The cutting can be collected and planted during any point in a year. Stated another way, the cutting can be collected and planted year round. In certain embodiments, the cutting is collected and planted in May or June. In other embodiments, the cutting is collected and planted in September, October, or November. In different embodiments, the cutting is collected and planted at the end of the dry season. In some embodiments, the cutting is collected and planted at the beginning of the rainy season. In the mountains of Peru, the wet season is typically November to mid-April, with the most rain received in January and February. In the mountains of Peru, the dry season is typically mid-April to October, with hot, dry days and cold, dry nights, often hovering just above freezing, particularly in June and July. In the jungles of Peru, the wet season is typically November to March, with hot and humid days and heavy rain expected at any time. In the jungles of Peru, the dry season is April to October. In the rainy season, the rain fall totals about 3 to about 6 inches per month. In the dry season, the rain fall totals about 0.1 to about 2 inches per month.

The cuttings are planted and grown under conditions suitable to grow V. pompona. Conditions suitable to grow V. pompona can include one or more of the following: warm and humid conditions such as an annual rainfall of about 150 to about 350 cm, a temperature range of about 25° C. to about 34° C., and about 20% to about 60% shade, well-drained soils comprising high organic content with a pH of about 6.5 to about 7.5, support for growth such as living trees, wooden stakes, bamboo stakes, stone pillars, iron pipes, and/or pollination such as by hand or bees.

In still another aspect, the disclosure provides a method of cultivating Vanilla pompona. The method comprises growing a V. pompona plant adjacent to a tree, wherein the V. pompona plant and the tree are surrounded by an irrigation trench which is located at least 2 m and less than 8 m in any direction from the base of the V. pompona plant and the tree. The method of cultivating V. pompona can be done without pesticides, herbicides or growth stimulating hormones. A minimum of one to a maximum of four V. pompona plants can be grown adjacent to a tree. For example, one to four, one to three, one to two, two to four, two to three, or three to four V. pompona plants can be grown adjacent to a tree. Additionally, one, two, three, or four V. pompona plants can be grown adjacent to a tree.

Generally speaking, the tree should be able to grow in the same growing conditions as the V. pompona, has the ability to fix nitrogen in the soil, facilitates a soil pH of about 6.5 to about 7.5, and provides shade and support for the V. pompona plant. In a specific embodiment, the tree is from the Inga genus. Inga, common name shimbillo, pacay, and guaba, is a genus of small tropical, tough-leaved, nitrogen-fixing trees, subfamily Mimosodeae. Non-limiting examples of Inga species include: Inga adenophylla, Inga alba, Inga allenii, Inga amazonica L.Cardenas, Inga amboroensis, Inga andersonii, Inga approximate, Inga aptera, Inga arenicola, Inga augusti, Inga balsapambensis, Inga barbata Benth., Inga bella, Inga bicoloriflora, Inga bijuga, Inga blanchetiana, Inga bollandii, Inga bracteifera, Inga bullata, Inga bullatorugosa, Inga cabelo, Inga calantha, Inga calanthoides, Inga calcicola, Inga canonegrensis, Inga carinata, Inga caudate, Inga chiapensis, Inga coragypsea, Inga cordistipula Mart., Inga cuspidate, Inga cynometrifolia, Inga densiflora Benth., Inga dominicensis, Inga dwyeri, Inga edulis Mart. (I. vera Kunth, I. vera sensu Brenan), Inga enterolobioides, Inga exalata T.S.Elias, Inga exfoliate, Inga exilis, Inga extra-nodis, Inga fastuosa, Inga feuillei (I. edulis sensu auct.), Inga fosteriana, Inga gereauana, Inga golfodulcensis, Inga goniocalyx, Inga grazielae, Inga herrerae, Inga hispida, Inga interfluminensis, Inga ismaelis, Inga jaunechensis, lnga jimenezii, Inga lacustris, Inga lanceifolia, Inga latipes, Inga laurina (Sw.) Willd. (I. fagifolia (L.) Benth.), Inga lenticellata, Inga lentiscifolia, Inga leptantha, Inga leptingoides, Inga litoralis, Inga macarenensis, Inga macrantha, Inga maritime, Inga martinicensis, Inga megalobotrys, Inga mendoncaei, Inga microcalyx, Inga mortoniana, Inga mucuna, Inga multicaulis, Inga multinervis, Inga neblinensis, Inga oerstediana Benth., Inga paffida, Inga pedunculata, Inga platyptera, Inga pleiogyna, Inga pluricarpellata, Inga porcata, Inga portobellensis, Inga praegnans, Inga punctata Willd., Inga ruiziana G.Don (I. fagifolia G.Don), Inga saffordiana, Inga salicifoliola, Inga saltensis, Inga santaremnensis, Inga sapindoides Willd., Inga sarayacuensis, Inga sellowiana, Inga semialata (Veil.) C. Mart., Inga sessilis (Veil.) Mart., Inga silanchensis, Inga sinacae, Inga skutchii, Inga spectabilis Willd., Inga spiralis, Inga stenophylla, Inga suberosa, Inga suborbicularis, Inga tenuicalyx, Inga tenuiloba, Inga thibaudiana, Inga umbellifera (Vahl) DC., Inga unica, Inga vera Willd., and Inga yasuniana. In a specific embodiment, the tree is Inga edulis or Inga feuillei. Common names for Inga edulis or Inga feuillei include pacay, guaba, and shambillo.

The V. pompona can be cultivated using the waru waru agricultural technique. The waru waru technique comprises combining raised beds with irrigation trenches so as to prevent damage due to soil erosion during floods. The waru waru technique ensures both collecting of water (either fluvial water, rainwater or phreatic water) and subsequent drainage. Water uptake by the raised beds is through diffusion and capillary movements using water contained within the beds or supplied from the surrounding irrigation trenches. The soils are kept at an adequate moisture level to facilitate the cultivation of V. pompona plants. When filled with water, the irrigation trenches ensure a microclimate that acts as a buffer against night frosts and provides moisture during droughts, and drainage during the rainy season. The irrigation trenches also act as barriers to keep out crawling insect pests and maintain the fertility of the soil.

An irrigation trench is located at least 2 m and less than 8 m in any direction from the base of the V. pompona plant and the tree. For example, an irrigation trench can be located at least 2 m and less than 7 m, at least 2 m and less than 6 m, at least 2 m and less than 5 m, at least 2 m and less than 4 m, at least 2 m and less than 3 m, at least 3 m and less than 8 m, at least 3 m and less than 7 m, at least 3 m and less than 6 m, at least 3 m and less than 5 m, at least 3 m and less than 4 m, at least 4 m and less than 8 m, at least 4 m and less than 7 m, at least 4 m and less than 6 m, at least 4 m and less than 5 m, at least 5 m and less than 8 m, at least 5 m and less than 7 m, at least 5 m and less than 6 m, at least 6 m and less than 8 m, at least 6 m and less than 7 m, or at least 7 m and less than 8 m in any direction from the base of the V. pompona plant and the tree. Additionally, an irrigation trench can be located at about 2 m, about 3 m, about 4 m, about 5 m, about 6 m, about 7 m, or about 8 m in any direction from the base of the V. pompona plant and the tree. Further, an irrigation trench can be located at 2 m, 3 m, 4 m, 5 m, 6 m, 7 m, or 8 m in any direction from the base of the V. pompona plant and the tree.

The location of the irrigation trench facilitates proper spacing of the tree. The distance dictated by the irrigation trench allows for healthier tree and V. pompona plant growth yet still provides ample shade for the V. pompona plant. At full growth of the tree, the tree provides between about 20% and about 60% shade for the V. pompona plant. For example, at full growth of the tree, the tree provides between about 20% and about 55%, between about 20% and about 50%, between about 20% and about 45%, between about 20% and about 40%, between about 20% and about 35%, between about 20% and about 30%, between about 20% and about 25%, between about 25% and about 60%, between about 25% and about 55%, between about 25% and about 50%, between about 25% and about 45%, between about 25% and about 40%, between about 25% and about 35%, between about 25% and about 30%, between about 30% and about 60%, between about 30% and about 55%, between about 30% and about 50%, between about 30% and about 45%, between about 30% and about 40%, between about 30% and about 35%, between about 35% and about 60%, between about 35% and about 55%, between about 35% and about 50%, between about 35% and about 45%, between about 35% and about 40%, between about 40% and about 60%, between about 40% and about 55%, between about 40% and about 50%, between about 40% and about 45%, between about 45% and about 60%, between about 45% and about 55%, between about 45% and about 50%, between about 50% and about 60%, between about 50% and about 55%, or between about 55% and about 60% shade for the V. pompona plant. Additionally, at full growth of the tree, the tree provides about 20%, about 25%, about 30%, about 35%, about 40%, about 45%, about 50%, about 55%, or about 60% shade for the V. pompona plant. Further, at full growth of the tree, the tree provides 20%, 25%, 30%, 35%, 40%, 45%, 50%, 55%, or 60% shade for the V. pompona plant.

The volume of water needed for growth of the tree and V. pompona plant is dependent upon soil conditions and plant requirements, the latter of which may vary at different periods of growth. A skilled artisan would be able to determine the volume of water needed. Water can be supplied via hoses or via irrigation trenches. The irrigation trench is sufficiently deep to allow water flow and collection from rainfall, surface and/or phreatic sources. The irrigation trench can be between about 12 inches to about 30 inches deep and between about 6 inches to about 18 inches wide. For example, the irrigation trench can be about 12 inches to about 24 inches, about 12 inches to about 18 inches, about 18 inches to about 30 inches, about 18 inches to about 24 inches, or about 24 inches to about 30 inches deep. Additionally, the irrigation trench can be about 12 inches, about 18 inches, about 24 inches, or about 30 inches deep. Further, the irrigation trench can be 12 inches to 30 inches deep, 12 inches to 24 inches, 12 inches to 18 inches, 18 inches to 30 inches, 18 inches to 24 inches, or 24 inches to 30 inches deep. Still further, the irrigation trench can be 12 inches, 18 inches, 24 inches, or 30 inches deep. Regarding width, the irrigation trench can be between about 6 inches to about 18 inches, about 6 inches to about 12 inches, or about 12 inches to about 18 inches wide. Additionally, the irrigation trench can be about 6 inches, about 12 inches, or about 18 inches wide. Further, the irrigation trench can be between 6 inches to 18 inches, 6 inches to 12 inches, or 12 inches to 18 inches wide. Still further, the irrigation trench can be 6 inches, 12 inches, or 18 inches wide.

The method of cultivating V. pompona can further comprise trellis supports between trees. Trellis supports provide additional support between trees and can be used to connect V. pompona plants above irrigation trenches.

The method of cultivating V. pompona can further comprise bee hives. Accordingly, in still yet another aspect, the disclosure provides a sustainable system for growing Vanilla pompona. The system comprises cultivating V. pompona as described herein and further comprises placing bee hives close to the trees. Natural self-pollination of Vanilla pompona is not possible due to absence of specific pollinating agents. Generally, hand-pollination is utilized to pollinate the Vanilla pompona. Hand-pollination must be done in the morning when the flower is open and is tricky and time consuming thereby limiting the amount of V. pompona that can be cultivated. To facilitate the cultivation of V. pompona, the V. pompona plant is grown in the presence of bee hives. The use of bee hives as presently described overcomes the limitation of hand-pollination and greatly increases the amount of V. pompona that may be cultivated. The bee hives comprise bees that pollinate the V. pompona plant. Specifically, the bee hives comprise Aveja manza (colmena), Aveja pequeno (ramichi), Xylocopa africana (fabricius) species of bees. The bee hives can be placed near the V. pompona plants such that pollination occurs year round.

### (a) Curing

After cultivation of the V. pompona, the V. pompona seed pods are then harvested, typically Vanilla seed pods are ready for harvest in six to nine months after pollination. The seed pods are harvested one by one when they are fully-grown and as they begin to ripen. At this stage, the seed pods change their color from dark green to light green with yellow tinge tips. Generally speaking, there are a few methods for curing Vanilla. For a review of the vanilla curing process, see D. Havkin Frenkel & R. Dorn, Vanilla, Chapter 4 in Spices: Flavor Chemistry and Antioxidant Properties, (Eds. Risch & Ho), American Chemical Society, Washington, 1997.

In one embodiment, the V. pompona seed pods are cured using the Silvestre method, by allowing the V. pompona seed pods to naturally cure on the vine. In another embodiment, the V. pompona seed pods are picked and spread in the hot sun on a black tarp then rolled up and taken in at night. This process is repeated until the vanilla beans are fully cured. In a preferred embodiment, shade curing is used. Shade curing is unique to V. pompona seed pods and is not found in other curing methods for the other two vanillas (V. planifolia or V. tahitensis). In this embodiment, after the V. pompona seed pods are picked, they are cured in the shade under ambient conditions until they are completely cured, this process can take anywhere up to 3 months for the seed pods to cure.

### II. Products

After cultivation and curing of the V. pompona, the V. pompona seed pods can be used for several downstream products. The V. pompona seed pods can first be cured prior to downstream processing of the cured V. pompona seed pods. Non-limiting examples of products that can be produced from the cultivated V. pompona include vanilla extract, vanilla sugar, vanilla syrup, vanilla oil, vanilla salt, vanilla salt and pepper, vanilla pepper, vanilla resin, vanilla absolute and vanilla paste. The vanilla products can be used for fragrance, food, cosmetic or dermatological compositions. In an embodiment, vanilla extract is produced by extracting cured V. pompona seed pods. In another embodiment, vanilla sugar is produced by combining sugar and spent V. pompona seed pods. In still another embodiment, vanilla syrup is produced by extracting cured V. pompona seed pods. In still yet another embodiment, vanilla oil is produced by extracting cured V. pompona seed pods. In other embodiments, vanilla paste is produced by extracting a paste from spent V. pompona seed pods. In still other embodiments, vanilla paste is produced by extracting a paste from cured V. pompona seed pods. In other embodiments, vanilla resin is produced by extracting a resin from V. pompona seed pods using a CO₂ extraction method. In still another embodiment, vanilla absolute is produced by CO₂ extraction of a resin produced from V. pompona seed pods.

The extraction step is usual in the plant extract field, and those skilled in the art are able to adjust the reaction parameters thereof, on the basis of their general knowledge, such as, for example, as detailed in Langenheim, Plant Resins: Chemistry, Evolution, Ecology, and Ethnobotany, Timber Press, Inc. 2003; Shinholt, Liquid Carbon Dioxide Extraction of Various Food Flavors: Evaluation and Analysis, Undergraduate Honors Thesis Collection. 48 (2009); and Kerrola, Literature review: Isolation of essential oils and flavor compounds by dense carbon dioxide, Food Reviews International Vol. 11, Iss. 4, 1995. Suitable extractions maybe single fold, double fold or triple fold as known in the art.

In a different embodiment, vanilla salt is prepared by mixing spent V. pompona seed pods with an edible salt. In certain embodiments, a vanilla salt and pepper preparation is produced by mixing spent V. pompona seed pods with an edible salt and pepper. In other embodiments, a vanilla pepper preparation is produced by mixing spent V. pompona seed pods with an edible pepper. Non-limiting examples of types of edible salt to be included in the vanilla preparation include kosher salt, crystalline sea salt, flaked sea salt, fleur de sel, rock salt, pickling salt, Himalayan pink salt, Celtic salt, sel gris, gros sel, Maldon salt, table salt, Hawaiian sea salt, smoked salt, seasoned salt, and black salt, and combinations thereof. Non-limiting examples of types of edible pepper to be included in the vanilla preparation include black pepper, green pepper, white pepper, pink or rose pepper, red pepper, cayenne pepper, and Szechuan pepper, Aji pepper, and combinations thereof.

The downstream products produced from the V. pompona seed pods cultivated according to the methods of the invention can be used for ingredients and components in a variety of compositions and products. In non-limiting examples, such compositions and products include, edibles (e.g. food and beverages), cosmetics (e.g. soaps, creams, and lotions), and fragrances. The downstream products produce from V. pompona of the present invention may further be blended with a flavor and/or fragrance component that is usually used by those skilled in the art, according to the intended purpose or usage. Examples of other flavor and/or fragrance components include various synthetic aroma chemicals, natural essential oils, synthetic essential oils, citrus oils and animal flavor and/or fragrance materials. For example, a wide range of flavor and/or fragrance components described in Arctander S. “Perfume and Flavor Chemicals” published by the author, Montclair, N. J. (U.S.A.), 1969 can be used.

Typical examples among these flavor and/or fragrance components include α-pinene, limonene, cis-3-hexenol, phenylethyl alcohol, styrallyl acetate, eugenol, rose oxide, linalool, benzaldehyde, methyl dihydrojasmonate and Thesaron (manufactured by Takasago International Corporation).

For example, when a natural essential oil such as bergamot oil, galbanum oil, lemon oil, geranium oil, lavender oil or mandarin oil is blended with one or more products that can be produced from the cultivated V. pompona according to the invention that makes an odor and the odor inherent to the natural essential oil to be mild, rich and fresh to give high preference, and that enhances diffusion property and retention property to give sustainability can be prepared.

Additionally, one or more products that can be produced from the cultivated V. pompona according to the invention can be blended with one or more commonly used flavor and/or fragrance retaining agents. Specific examples of such flavor and/or fragrance retaining agents include ethylene glycol, propylene glycol, dipropylene glycol, glycerin, hexyl glycol, benzyl benzoate, triethyl citrate, diethyl phthalate, Hercolyn (methyl abietate), and medium chain fatty acid triglyceride.

A product that can be odored using one or more products that can be produced from the cultivated V. pompona according to the invention is not particularly limited as long as the product needs to be added with such odor or flavor. Preferable examples include: fragrances and cosmetics such as fragrance products, basic cosmetics, makeup cosmetics, hair-care cosmetics and sunscreen cosmetics; quasi-drugs such as pharmaceutical cosmetics; toiletry products such as hair-care products, soap, body cleaning agents and bath agents; and goods such as detergents, fabric softeners, cleaning agents, kitchen detergents, bleaching agents, aerosols, deodorants/air fresheners, sanitary products and stationery.

Non-limiting examples of fragrance products include perfume, eau de parfum, eau de toilette and eau de cologne.

Non-limiting examples of basic cosmetics include face washing cream, vanishing cream, cleansing cream, cold cream, massage cream, emulsion, skin lotion, liquid cosmetics, pack and makeup remover.

Non-limiting examples of makeup cosmetics include foundation, face powder, pressed face powder, talcum powder, lipstick, lip cream, blush, eyeliner, mascara, eye shadow, eyebrow pencil, eye pack, manicure and nail enamel remover.

Non-limiting examples of hair-care cosmetics include pomade, brilliantine, hair setting lotion, hair stick, hair solid, hair oil, hair treatment, hair cream, hair tonic, hair liquid, hair spray, bandoline, hair growing agents and hair dyeing agent.

Non-limiting examples of sunscreen cosmetics include suntan products and sunscreen products. Examples of pharmaceutical cosmetics include antiperspirant, after-shave lotion and gel, permanent wave agent, medicated soap, medicated shampoo and medicated skin cosmetics.

Non-limiting examples of hair-care products include shampoo, hair rinse, two-in-one shampoo, hair conditioner, hair treatment and hair pack.

Non-limiting examples of soap include cosmetic soap, bath soap, perfumed soap, transparent soap and synthetic soap.

Non-limiting examples of body cleaning agents include body soap, body shampoo, hand soap and face cream.

Non-limiting examples of bath agents include bath additives (bath salt, bath tablet, bath liquid, etc.), foam bath (bubble bath, etc.), bath oil (bath perfume, bath capsule, etc.), milk bath, bath jelly and bath cube.

Non-limiting examples of detergents include heavy-duty laundry detergent, light-duty laundry detergent, liquid detergent, laundry soap, compact detergent and powdery soap.

Non-limiting examples of fabric softeners include softener and furniture care.

Non-limiting examples of cleaning agents include cleanser, household cleaner, toilet cleaning agents, bathroom cleaning agents, glass cleaner, mold removing agents and drain-pipe cleaning agents.

Non-limiting examples of kitchen detergents include kitchen soap, synthetic kitchen soap and dishwashing detergents.

Non-limiting examples of bleaching agents include oxidized bleaching agents (chlorine-based bleaching agents, oxygen-based bleaching agents, etc.), reduced bleaching agents (sulfur-based bleaching agents, etc.) and optical bleaching agents.

Non-limiting examples of aerosols include spray types and powder sprays.

Non-limiting examples of deodorant/air freshener include solid types, gel types and liquid types (water-based, oil-based).

Non-limiting examples of sanitary products include tissues and toilet paper.

Non-limiting examples of stationery include eraser, pencil, notebook and seal.

The dosage form of the one or more products that can be produced from the cultivated V. pompona according to the invention is not particularly limited. Any dosage form may be selected and used according to the purpose, including, for example: liquid forms dissolved in alcohols, polyalcohols such as propylene glycol, glycerin and dipropylene glycol, or esters such as triethyl citrate, benzyl benzoate and diethyl phthalate; emulsion forms emulsified with emulsifiers such as glycerin fatty acid ester and sucrose fatty acid ester; powder forms coated with excipients such as natural gummy matters (gum arabic, tragacanth gum, etc.), gelatin and dextrin; and solubilized or dispersed forms that are solubilized or dispersed with surfactants (for example, nonionic surfactants, anion surfactants, cationic surfactants, amphoteric surfactants, etc.).

Alternatively, any dosage form, such as a microcapsule form obtained by treating with a capsulating agent, may be used according to the purpose.

Moreover, the one or more products that can be produced from the cultivated V. pompona according to the invention can be stabilized and made to have sustained releasability by clathrating in a clathrate agent such as cyclodextrin. This is suitable when the form of the end product is, for example, a liquid form, a solid form, a powdered form, a gel form, a mist form or an aerosol form, which may appropriately be selected and used according to the form of the end product.

Some embodiments of the present invention provide for vanilla-flavored edible compositions described above. The vanilla-flavored compositions generally encompass any edible or consumable compositions, such as foods and beverages. More particularly, the vanilla-flavored compositions may be selected from forms such as, but not limited to, hard candy, soft candy, cotton candy, pressed tablets, chewing gum, film, lozenges, liquid beverages, powdered beverages, and the like. Such vanilla-flavored compositions include any of the vanilla-flavored compositions described above, a carrier and optionally additives, such as flavor agents, coloring agents and the like.

### (a) Methods of Producing Products

The methods of the present invention allow for the production of a variety of downstream products produced from the V. pompona plants cultivated according to the methods of the invention. The downstream products include, but are not limited to, a vanilla extract, a vanilla sugar, vanilla syrup, vanilla oil, a vanilla salt, a vanilla salt and pepper, a vanilla pepper, a vanilla resin, a vanilla absolute and a vanilla paste. The downstream products are produced and extracted according to techniques appreciated by those skilled in the art, according to the intended purpose or usage. For example, extraction may be carried out by using a solvent or CO₂ depending on the desired product produced.

In one embodiment, the present invention provides a method for producing a vanilla extract, the method comprising extracting cured V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

In another embodiment, the present invention provides a method of producing a vanilla sugar the method comprising combining sugar with spent V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

In yet another embodiment, the invention provides a method for producing a vanilla syrup, the method comprising extracting cured V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

The present invention further provides a method for producing a vanilla oil, the method comprising extracting cured V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

In one aspect, the present invention provides a method for producing a vanilla salt and pepper preparation, the method comprising mixing an edible salt and pepper with spent V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In another aspect, the present invention provides a method for producing a vanilla salt preparation, the method comprising mixing an edible salt with spent V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees. In still another aspect, the present invention provides a method for producing a vanilla pepper preparation, the method comprising mixing an edible pepper with spent V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

The present invention provides a method of producing a vanilla resin, the method comprising extracting a resin from V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees. The present invention further provides a method for producing a vanilla paste, the method comprising extracting a paste from spent V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

In one embodiment, the present invention provides a method of producing a vanilla absolute, the method comprising extracting an vanilla absolute from V. pompona seed pods cultivated by growing a V. pompona plant adjacent to a tree, wherein the V. pompona plant and the tree are surrounded by an irrigation trench which is located at least 2 m and less than 8 m in any direction from the base of the V. pompona plant and the tree. In one aspect, the method further comprises placing bee hives close to the trees.

In another embodiment, the present invention provides a method of producing a cosmetic, the method comprising blending a cosmetic composition with one or more of the downstream products produced from the V. pompona plant cultivated according to the invention. In some aspects, the methods include producing basic cosmetics, makeup cosmetics, hair-care cosmetics and sunscreen cosmetics; quasi-drugs such as pharmaceutical cosmetics; toiletry products such as hair-care products, soap, body cleaning agents and bath agents.

In yet another embodiment, the present invention provides a method of producing a fragrance, the method comprising the method comprising blending a fragrance composition with one or more of the downstream products produced from the V. pompona plant cultivated according to the invention.

In still another embodiment, the present invention provides a method of producing an edible, the method comprising blending an edible composition with one or more of a vanilla extract, a vanilla sugar, a vanilla syrup, a vanilla oil, a vanilla salt, a vanilla salt and pepper, a vanilla pepper, a vanilla paste, a vanilla resin or a vanilla absolute produced from the V. pompona plant cultivated according to the invention.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Cultivation of Vanilla pompona

A Vanilla pompona cutting between about 150 cm and about 250 cm long or with a minimum of 12 nodes is planted within 7 days following cutting. If the cutting is not planted immediately, then it is kept moist. For example, by wrapping in a damp paper towel. Leaves from the bottom couple nodes are then removed from the base of the cutting and the base is planted into loose soil. The soil is then packed around the nodes and a support for growth is placed adjacent to the cutting. The soil is kept damp until the cutting begins to show net growth. The cutting is grown in warm and humid conditions with about 60% shade. The soil has a high organic content with a pH of about 6.5 to about 7.5.

Four V. pompona cuttings planted as described above are placed around a support for growth such as an Inga tree. Beneficially, the tree provides support, shade, and fixes nitrogen for the V. pompona plant. Surrounding the Inga tree are trenches for irrigation. The trenches are about 5 m from the tree in any direction and about 12 to 30 inches deep and about 6 to 18 inches wide. The trenches ensure both collecting of water and subsequent drainage.

To eliminate or reduce the need for the tedious task of hand-pollination, bee hives are placed in the vicinity of the V. pompona plants. The bee hives house Aveja manza (colmena) or Aveja pequeno (ramichi) species of bees which can pollinate the V. pompona year round.

The vanilla beans are then harvested, typically when the tip starts to turn yellow or the whole been has turned yellow, and is then cured. One method to cure the vanilla beans is to dry them in the sun for about a month. Another method is to let the beans dry naturally on the vine, which can take several months. Yet another method is sombra curing, whereby the vanilla bean is cured in the shade under ambient conditions for a period of 22 days to 45 days. Still another method of curing is oven-wilting. The cured vanilla bean is then processed to make various vanilla products.

1. A method of transplanting Vanilla pompona to produce pods within the first year of growth, the method comprising collecting a cutting between about 150 cm and about 250 cm long, and planting said cutting under conditions suitable to grow V. pompona.

2. The method of claim 1, wherein the cutting has a minimum of 12 nodes.

3. The method of claim 1, wherein the cutting is planted within 7 days following cutting.

4. The method of claim 1, wherein the V. pompona is grown plant adjacent to a tree, wherein the V. pompona plant and the tree are surrounded by an irrigation trench which is located at least 2 m and less than 8 m in any direction from the base of the V. pompona plant and the tree.

5. A method of cultivating Vanilla pompona, the method comprising growing a V. pompona plant adjacent to a tree, wherein the V. pompona plant and the tree are surrounded by an irrigation trench which is located at least 2 m and less than 8 m in any direction from the base of the V. pompona plant and the tree.

6. The method of claim 5, wherein a minimum of one to a maximum of four V. pompona plants are grown adjacent to the tree.

7. The method of claim 5, wherein the tree grows in the same growing conditions as the V. pompona.

8. The method of claim 5, wherein the tree fixes nitrogen in the soil.

9. The method of claim 5, wherein the tree facilitates a soil pH of about 6.5 to about 7.5.

10. The method of claim 5, wherein the tree provides shade and support for the V. pompona plant.

11. The method of claim 5, wherein the tree provides between about 20% and about 60% shade for the V. pompona plant at full growth of the tree.

12. The method of claim 5, wherein the tree is a tree from the Inga genus.

13. The method of claim 5, wherein the tree is Inga edulis or Inga feuillei.

14. The method of claim 5, wherein the cultivating is via the waru waru agricultural technique.

15. The method of claim 5, wherein the irrigation trench is between about 12 inches to about 30 inches deep and between about 6 inches to about 18 inches wide.

16. The method of claim 5, wherein the method further comprises trellis supports between trees.

17. (canceled)

18. The method of claim 5, wherein the cultivating is done without pesticides, herbicides or growth stimulating hormones.

19. A sustainable system for growing Vanilla pompona, the system comprising cultivating the V. pompona according to claim 5 and further comprises placing bee hives close to the trees.

20. A method for curing V. pompona, the method comprising placing the V. pompona seed pod obtained from a V. pompona cultivated in accordance with claim 5 in the shade under ambient conditions for a period of about 22 days to about 45 days.

21. A method for producing a one or more of a vanilla extract, a vanilla syrup, a vanilla oil, a vanilla resin, a vanilla paste, or a vanilla absolute, the method comprising extracting cured V. pompona seed pods cultivated according to the method of claim 5.

22.-33. (canceled)
